(19) United States
(12) Patent Application Publication
(10) Pub. No.: US 2007/0270633 A1
(43) Pub. Date: Nov. 22, 2007

(54) Blood Pump Comprising Polymeric Components

(76) Inventors: Martin Christopher Cook, Coogee (AU); Natalie James, Mosman (AU); Naoki Fujisawa, Lindfield (AU)

Correspondence Address: MCCARTER & ENGLISH , LLP STAMFORD OFFICE, FINANCIAL CENTRE , SUITE 304A, 695 EAST MAIN STREET, STAMFORD, CT, 06901-2138, US

(21) Appl. No.: 10/577,563

(22) PCT Filed: Oct. 28, 2004
(86) PCT No.: PCT/AU04/01490

§ 371 Date: Mar. 27, 2007

(30) Foreign Application Priority Data
Oct. 31, 2003 (AU) 2003906051

(51) Int. Cl. A61M 1/12

(52) U.S. Cl. 600/16

(57) ABSTRACT

A rotary blood pump (13) including: a motor adapted to magnetically rotate an impeller (2) within a housing (6). The impeller and/or the housing are formed of a composite material and the composite material includes a first material that is a relatively, insulative, biocompatible and impermeable polymer. The composite material may include a second material that reinforces the polymer.

### FIELD OF INVENTION

The present invention relates to an improved implantable blood pump comprising polymeric components.

### BACKGROUND OF INVENTION

Previously, congestive heart failure may have been treated with the use of blood pump to assist the pumping of blood around the circulatory system of a patient.

U.S. Pat. No. 6,609,883—Woodard et al describes a blood pump fabricated mainly from Titanium-6 Aluminum-4 Vanadium (Ti-6A1-4V) coated with amorphous carbon and/or diamond-like coatings. In particular, the pump housing of this blood pump is metallic and includes a magnetic drive motor acting on a hydrodynamic impeller within the pump housing. One of the disadvantages with this invention is that as the pump housing is entirely constructed of metal, electrical eddy currents form between the motor stators and permanent magnets positioned within the impeller. These electrical eddy currents significantly reduce the electrical efficiency of the blood pump and may lead to increased power consumption.

Another U.S. Pat. No. 6,158,984—Cao et al describes a modified blood pump in which structural members are inserted within the pump housing between the motor stators and the impeller. These structural members are constructed of a biocompatible, corrosion resistant, electrically non-conductive (insulative) ceramic material. One of the disadvantages with the structural members being comprised of ceramic material is that ceramic material is relatively expensive and difficult to construct. The ceramic material may include a diamond like coating which may be particularly costly to produce and prone to flaking.

It has been previous known to this field, that rotary blood pumps may be entirely constructed from polymeric material except for the motor components. However, pumps that are entirely constructed of polymeric materials may lack the desired: wear resistance or strength, fluid impermeability and bio-resistance necessary for this type of application. These types of pumps commonly warp or distort due to fluid absorption limiting their usefulness.

It is an object of the present invention to address or ameliorate one or more of the abovementioned disadvantages.

### BRIEF DESCRIPTION OF THE INVENTION

In accordance with a first aspect the present invention consists in a rotary blood pump including: a motor adapted to magnetically rotate an impeller within a housing; characterised in that the impeller or the housing are formed of a composite material and said composite material includes a first material that is a relatively, insulative, biocompatible and impermeable polymer.

Preferably the composite material includes a second material that reinforces the polymer.

Preferably the pump includes an insulative member formed from said first material.

Preferably said insulative member is disposed between portions of the motor to reduce eddy currents losses.

Preferably said first material has been surface modified by treatment of plasma immersion ion implantation.

Preferably said impeller includes magnets that are encapsulated by an impermeable fluid barrier.

Preferably said first material is: PEEK, FRP, PC, PS, PEPU, PCU, SiU, PVC, PVDP, PE, PMMA, ABS, PET, PA, AR, PDSM, SP, AEK, T, MPP or a combination thereof.

Preferably said impeller is hydrodynically suspended.

In accordance with a second aspect the present invention consists in a rotary blood pump including: a motor adapted to magnetically rotate a hydrodynamically suspended impeller within a housing; characterised in that the impeller and/or the housing are formed of a composite material, said pump including at least one insulative member disposed between portions of said motor to reduce eddy current losses and said insulative member is substantially formed from a biocompatible and impermeable polymer.

Preferably said composite material includes a metal metallic alloy.

Preferably said metallic alloy is a titanium alloy.

### BRIEF DESCRIPTION OF DRAWINGS

Embodiments of the invention will now be described with reference to the drawings in which:

FIG. 1 shows a cross-sectional view of a first preferred embodiment of the present invention;

FIG. 2 shows an enlarged cross sectional view of a portion of the preferred embodiment shown in FIG. 1; and

FIG. 3 shows an enlarged rotated top view of a portion of the preferred embodiment shown in FIG. 1.

### DETAILED DESCRIPTION OF EMBODIMENTS

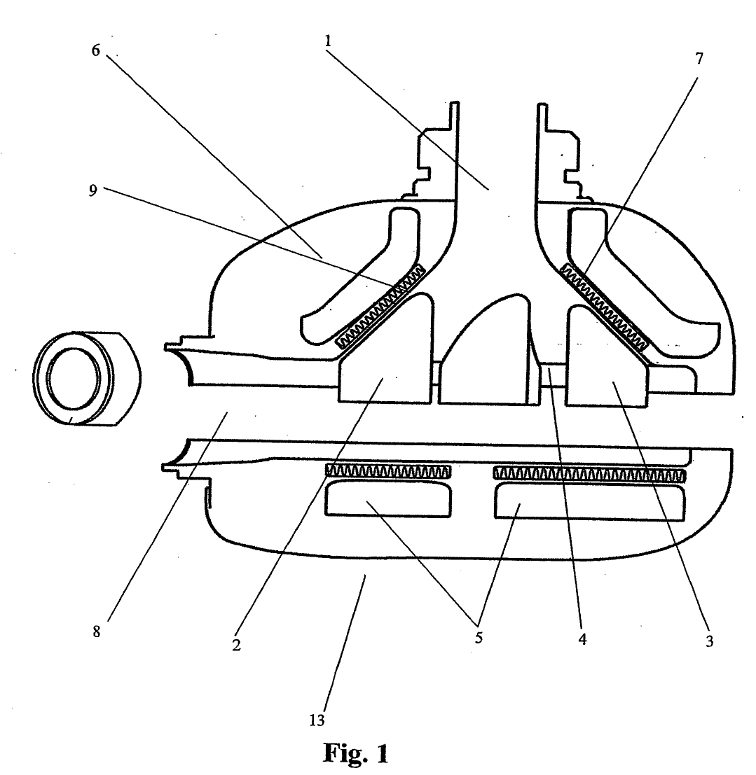

A first embodiment of the present invention is shown in FIG. 1. In this embodiment, a blood pump 13 made of a composite material, wherein the composite material includes at least a portion of polymer material reinforced with a second material which may preferably be titanium alloy or other wear resistance and biocompatible material. This blood pump 13 may include: an inlet 1 and an outlet 8; an impeller 2 which rotates and propels blood from the inlet 1 using centrifugal propulsion through the pump housing 6 to the outlet 8; a motor generates the torque force for rotating the impeller 2, the motor is formed by the interaction of the stators 5 axially mounted within the pump housing 6 interacting with magnetic regions in the impeller 2.

Preferably, the impeller 2, in use, is hydrodynamically suspended on a fluid bearing formed by a restriction gap 9 between the blades 3 of impeller 2 and the inner wall of the pump housing 6. The impeller 2 preferably includes four blades 3 joined together by struts 4 in a generally square configuration.

Preferably positioned between the stators 5 and the magnetic regions of the impeller 2 is an insulative member 7. This insulative member 7 is electrically non-conductive and may be constructed of polymers. The insulative member 7 functions so as to prevent or minimise the build up of electrical eddy currents between the stators 5 and magnetic regions of the impeller 2. The eddy currents interfere with the transfer of EMF onto the impeller 2 and may lead to a reduction of electrical efficiency. Once the eddy currents are reduced or minimised, the efficiency of the motor is greatly improved. This insulative member 7 may be encapsulated within the housing 6, as shown in FIG. 1, or embedded within the inner wall of the housing 6.

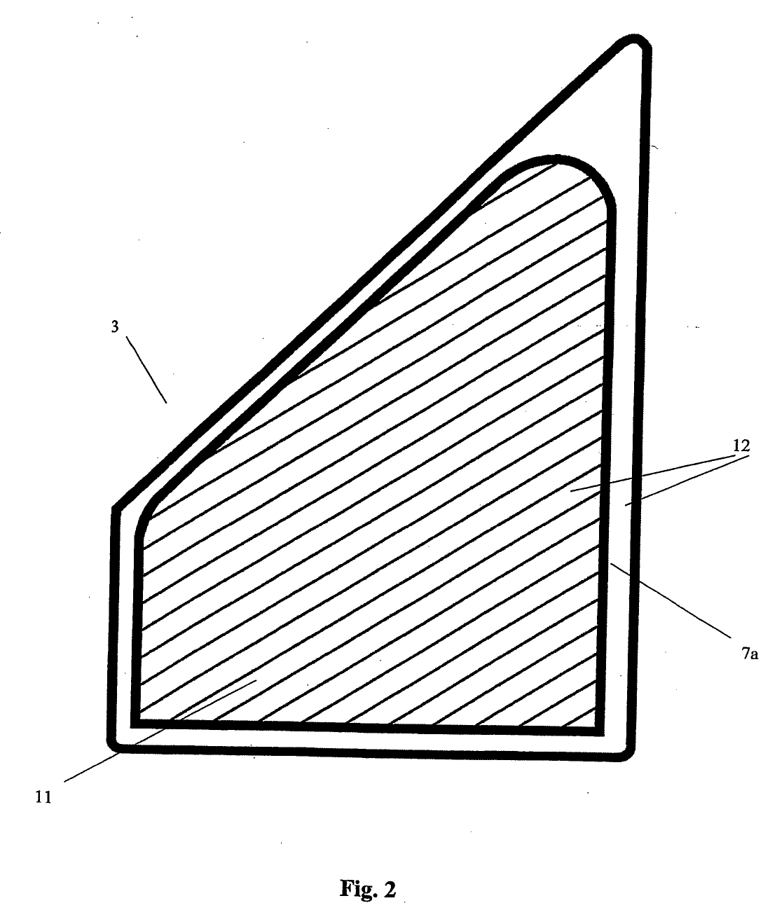

Additionally, FIG. 2 shows a cross sectional view of a blade 3. Generally, this blade 3 is made or constructed of a polymeric material. This polymeric material is shown as a layer which forms an insulative member 7a around the outer surface of the blade 3. Encapsulated within the blade 3 is a permanent magnet 11 surrounded by the insulative member 7a. As permanent magnets 11 may be generally comprised of bio-toxic compounds, it may be necessary to prevent the bio-toxic material from contacting the blood in the pump 13, when in use.

Most polymeric materials are at least partially susceptible to fluid permeation and as such bio-toxic compounds may degrade and release toxic chemicals or compounds in a patient's circulatory system. Therefore, it may be also preferable to coat the insulative member 7a in an impermeable barrier 12 to block, stop or greatly impede the eluting or release of bio-toxic compounds or chemicals into the patient's blood stream. The barrier 12 may also preferably encapsulate, coat and seal the permanent magnet 11.

Preferably, these barriers 12 may be constructed from gold, zinc, Paralene™ or similar impermeable coating material. Additionally the insulative member 7 may be surface modified so as to confer to the surface of the insulative member properties such as impermeability to fluids. These barriers 12 may be usable in any embodiment wherein the insulative member 7 is required to be sealed from the environment.

The insulative members 7 and 7a may be surface modified by plasma immersion ion implantation which may chemically alter the surface of the insulative members 7 and 7a to increase their hardness, durability and impermeability to fluids.

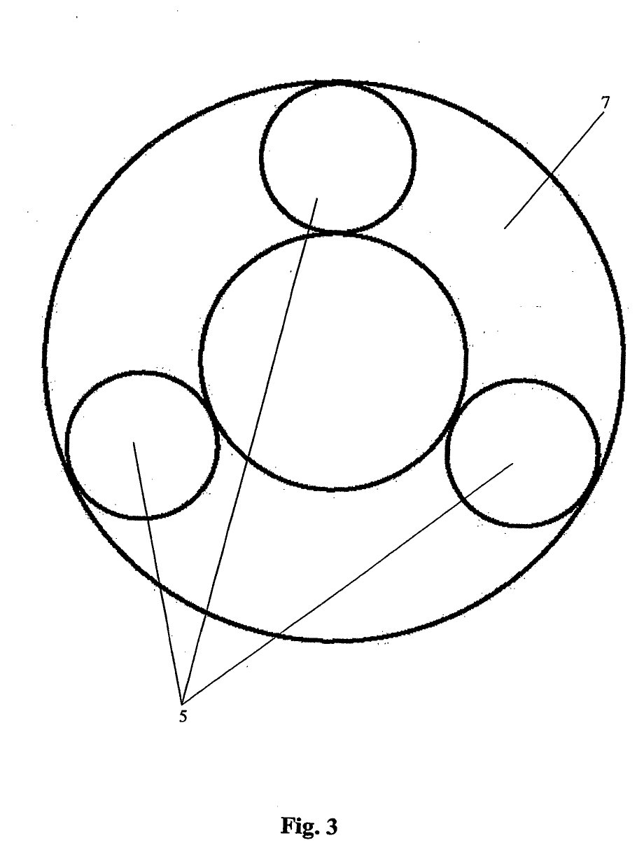

In FIG. 3, an enlarged top view of a preferred insulative member 7 is shown. This figure depicts a relatively flat disc shaped insulative member 7 mounted with three coils of wire forming the motor stators 5. This relatively flat insulative member may be adapted to fit in the lower inner surface of the housing 6 shown in FIG. 1. Alternately, the insulative member 7 may be modified to form a general cone shape suitable for use within the upper inner surface of the housing 6.

The following polymeric substances are examples of materials from which the embodiments may be constructed.

Polyetheretherketone (‘PEEK’)

An example of a polymeric material that may be used in the constructions of an embodiment is PEEK. It has a relatively high thermal stability compared with other thermoplastics. It typically retains high strength at elevated temperatures, and has excellent chemical resistance (being essentially inert to organics, and has a high degree of acid and alkali resistance). It has excellent hydrolytic stability and gamma radiation resistance. Therefore PEEK may be readily sterilised by different routes. It also shows good resistance to environmental stress cracking. It generally has excellent wear and abrasion resistance and a low coefficient of friction PEEK may incorporate glass and/or carbon fibre reinforcements which may enhance the mechanical and/or thermal properties of the PEEK material.

PEEK may be easily processed on conventional extrusion and injection moulding equipment. Post-annealing and other processes obvious to a person skilled in the art may be preferable. A polyaromatic, semicrystalline polymer may also be used in construction of an embodiment.

Other examples of this polymer include: Polyaryletherketone (‘PAEK’) manufactured by Vicltrex and PEEK-OPTINMA LT™ which is a polymer grade with properties optimised for long-term implants. PEEK-OPTIMA LT™ is significantly stronger than traditional plastics currently available. Generally, PEEK may be able to withstand more aggressive environments and maintain impact properties over a broader range of temperatures than other polymers.

It has been shown that carbon fibre reinforced PEEK found to exhibit excellent resistance to a saline environment at 37° C. designed to simulate human body conditions.

PEEK includes the significant advantage of generally supplying dimensional stability, when in use.

Fibre Reinforced Polymer (‘FRP’)

Another example of a polymeric material that may be included within an embodiment of the present invention is FRP. FRPs are constructed of composites of PEEK and other polymers. PEEK may be reinforced with 30% short carbon fibres and which when subjected to saline soaking, was found to exhibit no degradation in mechanical properties. In contrast, a 30% short carbon fibre reinforced polysulphone composite has been found to show degraded mechanical properties due to the same saline soaking.

The fibre/matrix bond strength may significantly influence the mechanical behaviour of FRP composites. Interfacial bond strength durability is therefore particularly important in the development of FRP composites for implant applications, where diffused moisture may potentially weaken the material over time. Testing in physiologic saline at 37° C. showed that interfacial bond strengths in carbon fibre/polysulfone and carbon fibre/polyetheretherketone composites significantly decrease.

It should be noted that the fibre/matrix bond strength is known to strongly influence fracture behaviour of FRP composites.

Polyearbonate (‘PC’)

Another example of polymer material that may be used in the construction of a preferred embodiment are PC resins. PC resins are widely used where transparency and general toughness are sought.

PC resins are intrinsically amorphous due to the large bulky bis-phenol component. This means that the polymer has a significantly high free volume and coupled with the polar nature of the carbonate group, the polymer can be affected by organic liquids and by water. PC resins are not as resistant to extremes in pH as PEEK however they are at least partially resistant.

PC resins generally have very low levels of residual monomers and so PC resins may be suitable for blood pump construction. PC resins generally have desirable mechanical and thermal properties, hydrophobicity and good oxidative stability. PC resins are desirably used where high impact strength is an advantage. PC resins also generally confer good dimensional stability, reasonable rigidity and significant toughness, at temperatures less than 140° C.

PC resins may be processed by all thermoplastic processing methods. The most frequently used process is injection moulding. Please note that it may be necessary to keep all materials scrupulously dry due to small but not negligible moisture pick-up of this resin. The melt viscosity of the resin is very high, and so processing equipment should be rugged. Processing temps of PC resins are relatively high generally being between approximately 230° C. and 300° C.

Polysulphone (‘PS’)

Another example of a polymeric material that may be used to construct parts of an embodiment from is PS. PS has relatively good high temperature resistance, and rigidity. PC is generally tough but not notch-sensitive and is capable of use up to 140° C. It has excellent hydrolytic stability and is able to retain mechanical properties in hot and wet environments. PS is generally chemically inert.

PS is similar to PC resins but may be able to withstand more rigorous conditions of use. Additionally, PS is generally more heat resistant, and possesses a greater resistance to creep and better hydrolytic stability. PC has a high thermal stability generally due to bulky chemical side groups and rigid chemical main backbone chains. It is also generally resistant to most chemicals.

Injection moulding used for lower melt index grades, whilst extrusion and blow moulding is used to form components generally made of higher molecular weight PS.

Polyarethanes (PU)

Another example of a polymeric material that may be include within an embodiment of the present invention is PU. PU is one of the most biocompatible and haemocompatible polymeric materials. PU has the following properties: elastomeric characteristics; fatigue resistance; compliance and acceptance or tolerance in the body during healing; propensity for bulk and surface modification via hydrophilic/hydrophobic balance or by attachments of biologically active species such as anticoagulants or bio-recognisable groups. Bio-modification of PU may be possible through the use of a several antioxidants used in isolation or in combination. These antioxidants may include vitamin E, which may create materials which can endure in a patient's body for several years.

PU constitutes one of the few classes of polymers that include the properties of being generally highly elastomeric and biocompatible.

Polyether Polyurethanes (‘PEPU’)

Another polymeric material that may be used in the construction of an embodiment is PEPU. PEPU generally has: relatively good flexural performance and acceptable blood compatibility.

Polycarbonate Urethane (‘PCU’)

PCU may also provide another alternative polymeric material for the purpose of constructing an embodiment. PCU has significantly lower rates of water transmission or impermeability. This is due to inherently lower chain mobility of the carbonate structure in the soft segment phase. Additional impermeability to water vapour can be achieved by selecting a polyurethane polymer with high hard segment content, and aromatic rather than aliphatic di-isocyanate co-monomer, and a more hydrophobic surface.

PCU generally has oxidative stability of the carbonate linkage, which reduces the rate of biodegradation tremendously as compared to the polyether polyurethanes.

Siloxane-Urethanes (‘SiU’)

SiU is another example of an alternative preferred polymeric material. SiU generally has a combination of properties including: fatigue strength, toughness, flexibility and low interaction with plasma proteins. However these polymers may be relatively soft.

Polyvinylehloride (‘PVC’)

PVC is another example of an alternative preferred polymeric material. PVC is a relatively amorphous and rigid polymer which in the absence of plasticiser has a glass transition around Tg 75° C.-105° C. It is a cheap tough polymer which is extensively used with many types of filler and other additives. Although it has a high melt viscosity and therefore in theory is difficult to process, specialised methods have been established for several decades to compound this polymer efficiently.

Extraction-resistant grades of PVC are required for long-term blood compatibility. Plasticised PVC has been well established for blood bags and similar devices, and resin manufacturers can keep toxic residual monomer levels acceptably low (<1 ppm). However there is enormous social pressure to outlaw PVC despite scientific data which generally indicates that PVC is benign.

Poly Vinylidene Fluoride (‘PVDF’)

PVDF is a polymer that possesses relatively good amounts of toughness and biocompatibility to be suitable for use in constructing an embodiment.

Polyethylene (‘PE’)

PE is available in several major grades, including Low Density PE (‘LDPE’), High Density PE (‘HDPE’) and Ultra High Molecular Weight Grade PE (‘UHMWPE’). However the UHMWPE may be likely to be the most suitable as it generally possesses relative toughness, low moisture absorption, and good overall chemical resistance.

Sintered and compression moulded UHMWPE has been well established for hip joints replacement. However further improvements appear necessary, as abrasive resistance and wear are not suitable for lengthy (>5-10 year) use. A major limitation of PE is thermal performance (melting point approximately 130° C.) and dimensional stability.

Polypropylene (‘PP’)

Another suitable polymeric material is PP. PP is a versatile polymer that may possess a combination of features including: relative inertness, relatively good strength and good thermal performance. Depending on the grade, Tg ranges from 0° C. to −20° C. and the MPt is approximately 170° C. The most common grades are homo- and ethylene copolymers, the latter with improved toughness.

In addition, there have been many advances in reactor technology leading to grades which are either much softer than normal or much stiffer. For example, the Bassell Adstiff™ polymers made using Catalloy™ technology may be suitable and/or include desirable features for use in the manufacture of a blood pump. Generally, PP polymers lack the high melting point of PEEK, but this property is not generally desired.

Polymethylmethacrylate (PMMA)

PMMA is an amorphous material with good resistance to dilute alkalis and other inorganic solutions, and has been shown to be one of the most biocompatible polymers. Therefore, PMMA may include some of the desirable features and may be used in the construction of an embodiment of the present invention. Generally, PMMA easily machined with conventional tools, moulded, surface coated and plasma etched.

PMMA's may be susceptible to environmental stress cracking although this is usually associated with the use of organic solvents, not present in a patient's body and a blood pump working environment.

Acrylonitrile-Butadiene-Styrene Terpolymers (ABS)

ABS generally have relatively good surface properties including: hardness, good dimensional stability and reasonable heat resistance (Tg approximately 120° C.). The combination of the three monomers imparts stiffness (styrene), toughness (butadiene) and chemical resistance (acrylonitrile).

Other attributes of ABS may include: rigidity, high tensile strength and excellent toughness as well as excellent dimensional accuracy in moulding. ABS is generally unaffected by water, inorganic solvents, alkalies; acids; and alcohols. However certain hydrocarbon solvents, not usually present within the body of a patient or in the working environment of the blood pump, may cause softening and swelling on prolonged contact.

Polyesters (‘PET’)

PET have become one of the largest growing thermoplastics over the past decade: volumes and prices are now approaching PE and PP. PET has a Tg around 75° C. and melting point of 275° C. It can vary from about 25% to 70% in crystallinity depending on the processing history of the polymer. Physical properties and chemical resistance are very dependant on crystallinity. PET may also have limited dimensional stability, as crystallisation can slowly increase after moulding. PET are generally tough, transparent, stiff and opaque.

Another class of PET with a Tg above 100° C. is currently available, this polymer is called Polyethylene Naphthenate (‘PEN’). PET and PEN may both be suitable for use in the construction of a blood pump.

Polyamides and/or Nylons (‘PA’)

PAs and Nylons are characterised by having excellent wear/frictional properties, high tensile impact and flexural strength and stiffniess, good toughness and high melting points.

Some PAs may include relatively large hydrocarbon spacers between the amide groups. Examples of this type of PA include Nylon 11 and 12 which are generally more hydrophobic (water uptake <1%) than regular varieties of PAs. However the larger spacing leads to a loss in stiffness compared to the other polymers and thermal performance may also be compromised.

Fully aromatic polyamides including Kevlar™ (sara position) and Nomexn5 (meta position) are commercially available and have high stiffness and melting points. Semi-aromatic polyamides are made in Germany (eg Trogamid™ T) and France. These semi-aromatic polyamides generally have good transparency and chemical resistance.

Acetal Resins and/or Polyoxymethylene (‘AR’)

AR may be used to construct any one of the preferred embodiments. This class of polymer is strong, hard, and abrasion resistant. It has been evaluated for joint replacement components and other long-term implants.

The acetal homo-polymer is prone to salt induced cracking, but copolymers with small amounts of a propylene oxide are possible. AR which contains formaldehyde may be of concern due to possible toxicity of formaldehyde.

Polydimethylsiloxane (‘PDSM’)

PDSM may be used to construct any one of the preferred embodiments. This polymer is generally elastomeric. It may also be considered for use as either a biocompatible coating or a copolymer.

Copolymers based on PDMS and PU have been developed and PDMS/PC is commercially offered by General Electric as Lexan™ 3200. The latter is a fairly stiff transparent material with excellent UV performance.

Syndiotactic Polystyrene (‘SP’)

SP may be used to construct any one of the preferred embodiments. SP is typically highly crystalline, little change in modulus occurs at the Tg of 100° C., and retention of properties is fairly high to the melting point of over 250° C. Many grades may be fibre reinforced, to filer reduce the change in modulus at the Tg. Being a hydrocarbon with no hetero atoms, the polymer may be hydrophobic and inert.

Aliphatic ether ketones (‘AEK’)

AEK may be used to construct any one of the preferred embodiments. Processing and mechanical performance are similar, but this polymer shows improved high temperature aging behaviour and little notch sensitivity. Unfortunately the material lacked distinctiveness and is no longer produced.

TOPAS™ (‘T’)

T may be used to construct any one of the preferred embodiments. This class of co-polymer is made by Ticona in Geamany. It generally comprises ethylene and norbomadene, with the Tg being controlled by monomer ratio. It is a hydrocarbon alternative to polycarbonate, and is generally suitable for medical fittings and devices. Its Tg is over approximately 130° C. and it is generally transparent with the co-monomer inibiting crystallisation of the ethylene segments.

Metallocene PP (‘MPP’)

MPP may be used to construct any one of the preferred embodiments MPP is manufactured by Exxon to compete with existing PP. It has a much narrower molecular weight distribution (polydispersity around 2) because it is oligomer-free.

Various additional modifications are possible within the scope of the foregoing specification and accompanying drawings without departing from the scope of the invention.

1. A rotary blood pump including: a motor adapted to magnetically rotate an impeller within a housing; characterised in that the impeller or the housing are formed of a composite material and said composite material includes a first material that is a relatively, insulative, biocompatible and impermeable polymer.

2. The rotary blood pump as claimed in claim 1, wherein the composite material includes a second material that reinforces the polymer.

3. The rotary blood pump as claimed in claim 1, wherein the pump includes an insulative member formed from said first material.

4. The rotary blood pump as claimed in claim 3, said insulative member is disposed between portions of the motor to reduce eddy currents losses.

5. A rotary blood pump as claimed in claim 1, wherein said first material has been surface modified by treatment of plasma immersion ion implantation.

6. A rotary blood pump as claimed in claim 1, said impeller includes magnets that are encapsulated by an impermeable fluid barrier.

7. A rotary blood pump as claimed in claim 1, wherein said first material is: PEEK, FRP, PC, PS, PEPU, PCU, SiU, PVC, PVDF, PE, PMMA, ABS, PET, PA, AR, PDSM, SP, AEK, T, MPP or a combination thereof.

8. The rotary blood pump as claimed in claim 1, wherein said impeller is hydrodynamically suspended.

9. A rotary blood pump including: a motor adapted to magnetically rotate a hydrodynamically suspended impeller within a housing, characterised in that the impeller and/or the housing are formed of a composite material, said pump including at least one insulative member disposed between portions of said motor to reduce eddy current losses and said insulative member is substantially formed from a biocompatible and impermeable polymer.

10. A rotary blood pump as claimed in claim 9 wherein said composite material includes a metal metallic alloy.

11. A rotary blood pump as claimed in claim 10 wherein said metallic alloy is a titanium alloy.
